(19) United States
(12) Patent Application Publication
(10) Pub. No.: US 2002/0192171 A1
(43) Pub. Date: Dec. 19, 2002

(54) Method for preventing malodor

(76) Inventors: Weiwei Li, New York, NY (US); Min Liu, New York, NY (US)

Correspondence Address: Mr. Weiwei Li, 345 East 68th Street, #1B, New York, NY, 10021, US

(21) Appl. No.: 09/839,517

(22) Filed: Apr. 21, 2001

(51) Int. Cl. A61K 7/32; A61K 31/70

(52) U.S. Cl. 424/65; 514/23; 514/27; 514/62

(57) ABSTRACT

This invention involves a method for preventing malodor by topically applying to human skin an active agent capable of reducing transport of odor-producing precursors to skin surface through inhibiting the glycoprotein carrier of human odorant.

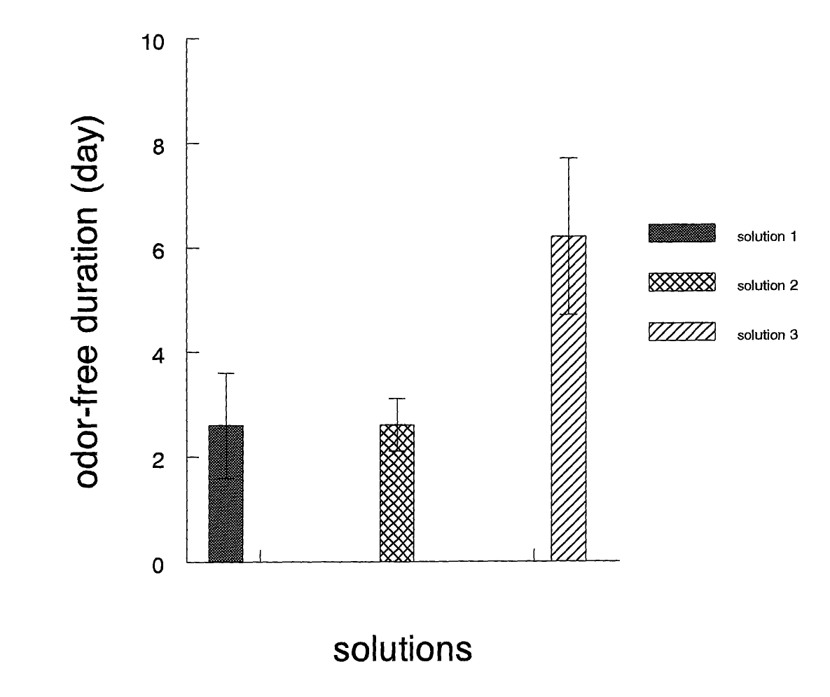

### CROSS-REFERENCE TO RELATED APPLICATIONS

Not applicable

### STATEMENT REGARDING FEDERALLY SPONSORED RESEARCH OR DEVELOPMENT

Not applicable

### REFERENCES CITED

### U.S. PATENT DOCUMENTS

| | | | |
|---|---|---|---|
| 4278658 | Jun., 1981 | Hooper et al | 424/65 |
| 5643559 | Jul., 1997 | Bigen et al | 424/67 |
| 5908867 | Jun., 1999 | Henry et al | 514/693 |

### OTHER REFERENCES

Labows J. N. et al: Steroid analysis of human apocrine secretion. Steroids 34: 249-258, 979.

Leyden J. J. et al: The microbiology of the human axilla and its relationship to axillary odor. J Invest Dermatol, 77: 413-416, 1981.

McGee T et al: The design principles of axilla deodorant fragrances. Ann NY Acad Sci 855: 841-6, 1998.

Toth I et al: Steroids excreted by human skin. II. C19-steroid sulphates in human axillary sweat. Acta Med Hunag. 42: 21-28, 1985.

Spielman A. I. et al: Proteinaceous precursors of human axillary odor: isolation of two novel odor-binding proteins. Experientia, 51: 40-47, 1995.

Zeng C et al: A human axillary odorant is carried by apolipoprotein D. Proc. Natl Acad Sci. USA 93: 6626-6630. 1996.

Patel S C et al: Astrocytes synthesize and secrete the lipophilic ligand carrier apolipoprotein D. Neuroreport 6: 653-7, 1995.

Teuter S et al: Ttransport of dehydroepiandrosterone and dehydroepiandrosterone sulphate into rat hepacytes. J. Steroid Biochem Mol Biol 54: 227-235, 1995.

Machamer C E et al: Monensin prevents terminal glycosylation of the N-and O-linked oligosaccharides of the HLA-DR-associated invariant chain and inhibits its dissociation from the alpha-beta chain complex. Proc. Natl. Acad. Sci. USA 81: 1287-1291, 1984.

### BACKGROUND OF THE INVENTION

Formation of body odor especially axillary odor mainly results from exceptional odor-producing abilities in the areas of body. These abilities include the following:

1) There are increased apocrine sweat glands in these areas, which are comprised of ducts that open directly into the hair follicle and become functional at puberty. It is known that there is a high level of 5alpha-reductase activity in human apocrine glands of which type-1 5alpha-reductase is predominant. 5alpha-reductase converts testosterone (T) to dihydrotestoserone (DHT) and may play a central role in the apocrine gland function from the beginning of puberty. The androgen receptor levels were also found to be high in the apocrine glands of patients with osmidrosis. These observations indicate that activity of androgens is involved with functions of apocrine sweat glands.

2) Sweat secreted from apocrine sweat glands contains numerous substances, which are most likely the precursors for forming odoriferous compounds, even although these precursors are odorless. These precursors mainly consist of (1) volatile odor-producing steroids such as dehydroepiandrosterone (DHEA), 5α-androst-16-en-3-one (androstenone) and 5α—androst-16-en-3α-ol (adrostenol); (2) nonvolatile steroid sulfates such as dehydroepiandrosterone sulphate (DHEAS), which can be microbially converted to odorous androstenol; (3) volatile short-chain fatty acids, especially (E)-3-methyl-2 hexenoic acid (E-3M2H), and cholesterol esters. It is demonstrated that E-3M2H is presented in far greater quantities than volatile odoriferous steroids (700:1) in apocrine secretions, although both have similar olfactory thresholds.

3) Odorless apocrine precursors are transported to skin surface by odorant carrier proteins. A main odor-producing precursor, E-3M2H, can be carried to the skin surface by binding to apolipoprotein D. Apolipoprotein D is expressed in apocrine sweat glands as an apocrine secretion odor-binding protein (ASOB2). ASOB2 is a glycoprotein and found to be glycosylated for its functions.

4) Odorless apocrine precursors carried to the skin surface interact with the microflora in the area of body such as the underarm to cause the characteristic odor.

Deodorant products in market are typically based on three principles to control body odor, especially in the underarm regions: (1) reduce perspiration; (2) inhibit bacterial growth and (3) cover malodor. These products show to have some effects on reducing unpleasant body odor. However they still have considerable disadvantages. First, effective period of these products is very limited. For example, an active component in commercial products for reducing perspiration is aluminum salt, which blocks the sweat gland ducts but only causes a 50% of perspiration reduction. Germicides in these products reduce the numbers of microorganisms on the surface of the local skin. However the effect obtained with the germicides are easily reversed by the rapid recovery of microflora number. Next, the active components in the products often cause irritation, burning, itching and other uncomfortable sensations to some people with sensitive skin. Lowering the amount of active components in the products may reduce irritation but it may also result in an impaired efficacy. Therefore, there remains a need for improved products. The present invention provides a novel method for preventing malodor by reducing transport of odor-producing precursors to skin surface.

### BRIEF SUMMARY OF THE INVENTION

The present invention is to provide a novel method by inhibiting apolipoprotein D, an odorant carrier protein to achieve the deodorant effect.

### BRIEF DESCRIPTION OF THE DRAWINGS

Figure is a graph of deodorant effect of 8.2% aluminum chlorohydrate (solution 1), 0.4% chlorhexidine digluconate (solution 2), or 0.4% monensin (solution 3). The test was carried out as described in Example 2.

### DETAILED DESCRIPTION OF THE INVENTION

The invention relates to a method for preventing malodor by inhibiting apoplipoprotein D, an odorant carrier protein.

Apolipoprotein D is a glycoprotein and its functions require the glycosylation. Glycoproteins are protein molecules, which are formed by attaching oligosaccharide chains to their polypeptide backbone structure with either O-or N-glycosidic linkage. Monensin is an ionophore commercially available and prevents the terminal glycosylation reaction by inhibiting N-linked oligosaccharide processing, thereby inhibiting the synthesis of glycoproteins. Monensin also inhibits transport of membrane glycoproteins and secretion proteins from Golgi apparatus. Therefore monensin can suppress the function of apolipoprotien D as an odorant carrier. Monensin was also found to reduce cholesterol uptake by inhibiting formation of cholesterol transport vesicle and to inhibit transport of dehydroepiandrosterone sulphate into cells.

The use of aluminum chlorohydrate in combination with odor-mask substances or anti-microbial agents for producing deodorant compositions is known from prior art. We have surprisingly discovered that monensin also provides a deodorant effect with a longer period than expected in preventing malodor. We further found that monensin did not cause any irritation at the concentrations used in this invention.

In this invention, the deodorant active material is dissolved in a cosmetically acceptable carrier to provide a deodorant composition to prevent malodor under armpit and/or other area of skin. The carrier material for the composition according to the present invention can comprise one or more volatile carrier fluids if required. In practice, the invention composition may preferably contain low molecular weight aliphatic alcohol such as ethanol. Ethanol proportion in the composition may be selected within the range of from 30-90% by weight. Other cosmetically acceptable carrier material can comprise a liquid or a mixture of fluids such as low molecular weight hydrocarbons and volatile low viscosity silicones, selected according to the physical form of the cosmetic vehicles. The cosmetic vehicles can be in the form of a fluid, cream, lotion, gel stick or spray, preferably in the form of a fluid.

The deodorant composition of the prevent invention will now be illustrated in detail by the following examples:

### EXAMPLE 1

The following is a typical formulation, which comprises an active agent in the form of a liquid deodorant according to the invention.

| Composition | |
|---|---|
| ingredient | % by weight |
| Monensin | 0.4 |
| Ethanol | 60 |
| Water | 39.6 |

### EXAMPLE 2

A typical test for deodorant effect of the present invention was carried out as follows. The test solution was the composition containing 0.4% monensin. The positive control was 8.2% aluminum chlorhydrate, a level that is used in most deodorant or antiperspirant products on the market and 0.4% chlorhesidine digluconate, a level that is usually used for bacterial killing. The negative control was a vehicle consisting of 60% ethanol and 40% water. Three experienced underarm odor assessors who are able to rank correctly the odor intensities based on the deodorant value test system (U.S. Pat. No. 4,278,658) performed odor assessments. Deodorant assessment scores were recorded according to the malodor category scale, when applied directly to the axillae skin of a panel of human subjects.

Malodor category Scale

| | | conc. of isovaleric |
|---|---|---|
| Score | description of odor | acid solution (ml/L) |
| 0 | no odor | 0 |
| 1 | slight | 0.013 |
| 2 | definite | 0.053 |
| 3 | moderate | 0.22 |
| 4 | strong | 0.87 |
| 5 | very strong | 3.57 |

The described in deodorant value test system has been modified by measuring odor intensity every 24 hours after treatment until odor intensity is detectable (average score 1 or up) on a 0-5 score instead of just 24 hours after

In example 2, fifteen subjects selected by experienced odor assessors have average odor score 3.5 (range from score 3-5). To avoid any product carry-over effects occur, all of subjects have not used any underarm products before the test for at least 10 days. Fifteen subjects were randomly divided into 3 groups (five subjects/each group) to test the composition and positive control products. Before testing, the subjects were instructed to take a shower in the evening using ordinary soap to clean the body surfaces. After the skin was dried the different products were topically tested in one axilla, versus the control vehicle in the other. The subjects then go about their normal business. At 24 hour intervals, each subject was evaluated for underarm odor by averaging the score of the three assessors until odor in test groups was detectable (score 1 or up). A summary of the results of the test is given in the figure. The solution containing monensin was more effective in preventing underarm odor than the positive control products. The composition of the present invention was also associated with no skin irritation in tested subjects.

What is claimed is:

1. A method for preventing malodor by topically applying to human skin an active agent which reduces transport of odor-producing precursors to skin surface by inhibiting human odorant carrier.

2. The method of claim 1, wherein said human odorant carrier is a glycoprotein.

3. The method of claim 1, wherein said human odorant carrier is an apolipoprotein D.

4. the method of claim 1, wherein said active agent inhibits the formation of glycoproteins.

5. The method of claim 1, wherein said active agent is selected from the group consisting of monensin, tunicamycin, amphmycin diumycin, showdomycin, tsushimycin, amphortericine, mycospocidin, streptovirudin, glucobeta-OH-norvaline, and D-glucosamine.

6.The method of claim 1, wherein said active agent is monensin in an amount of from 0.01% to 10% by weight of the composition.
